(19) United States
(12) Patent Application Publication
(10) Pub. No.: US 2022/0362093 A1
(43) Pub. Date: Nov. 17, 2022

(54) APPARATUS FOR TREATING SYMPTOMS ASSOCIATED WITH NEUROPATHY

(71) Applicant: MICELI ENTERPRISES, LLC, Palmetto, FL (US)

(72) Inventors: JEFFREY A. MICELI, SR., Palmetto, FL (US); JEFFREY A. MICELI, JR., Brentwood, TN (US)

(21) Appl. No.: 17/663,231

(22) Filed: May 13, 2022

Related U.S. Application Data
(60) Provisional application No. 63/189,290, filed on May 17, 2021.

(51) Int. Cl. A61H 3/00; A61H 1/02; A61B 5/11; A61H 39/00

(52) U.S. Cl. CPC A61H 3/00; A61H 1/0266; A61B 5/112; A61H 39/007; A61H 2201/5061; A61H 2201/165; A61H 2201/164

(57) ABSTRACT

An apparatus for reducing symptoms associated with neuropathy such as pain or other sensation deficits and sensation dysfunction in the lower extremity of a patient includes a platform having a base and a top plate supported by the base and configured to allow a patient to place their feet thereon. A vibrational actuator is carried by the base and connected to the top plate and configured to vibrate the top plate when a patient stands thereon or while seated with their feet placed on top of the plate. A controller is connected to the vibrational actuator and configured to operate the vibrational actuator to impart a vibrating force to the top plate when a patient has their feet thereon and stimulate the Pacini corpuscles to impart a neurological feedback response that dilates blood vessels of the feet and legs.

### PRIORITY APPLICATION(S)

This application is based upon U.S. provisional application Ser. No. 63/189,290 filed May 17, 2021, the disclosure which is hereby incorporated by reference in its entirety.

### FIELD OF THE INVENTION

This invention relates to treating symptoms commonly associated with neuropathy such as pain in a patient, and more particularly, to treating symptoms of neuropathy in a patient's feet as well as improving balance and increasing blood flow below the knee, including the ankles, feet, and toes.

### BACKGROUND OF THE INVENTION

Peripheral neuropathy is a debilitating condition that affects millions of people in the United States and worldwide. Peripheral neuropathy affects nerves that carry information to and from the brain and spinal cord to the rest of the body. In many cases, peripheral neuropathy affects feet and interferes with the cutaneous feedback from the feet that is essential for maintaining normal gait and balance. Cutaneous feedback is normally transduced via mechanoreceptors at the sole and transmitted via afferent nerve fibers to the central nervous system.

Peripheral neuropathy that affects the feet and other limbs may result in pain and a loss of sensation and inability to control muscles leading to the poor balance and gait and increased falls and foot ulcers. Most patients suffering from peripheral neuropathy, such as in the feet, are diabetics, oncology patients, autoimmune disease patients, and spinal cord injury patients. Other patients have unknown causes of peripheral neuropathy.

Patients suffering from peripheral neuropathy have a functional loss of nerve fibers that leads to gait and balance disorders and may increase the risk of developing foot ulcerations and can lead to amputations. Often patients have a purple color in their feet and lack sensation, making walking difficult. There are different rehabilitative techniques and remedies to alleviate peripheral neuropathy. Many doctors mandate dietary changes to treat diabetes, alcoholism or rheumatoid arthritis if they believe those factors are the underlying cause of the peripheral neuropathy. Medications are available, but can impact other systems of the body, or cause discomfort to the patient.

Some systems for treating neuropathy impart bias signals to sensory cells, such as on the feet. For example, one proposed system includes a wearable device that is secured around the calf of a patient to impart a vibrating motion. Other treatments may include implantable devices that impart vibration to certain muscles and nerve fibers. Some treatments include having a patient lie prone and elevate their legs onto a vibration generator that generates a counter-stimulation vibration that may help a patient treat their peripheral neuropathy. This same treatment may also help when a patient suffers from restless leg syndrome. Other stimulators may include devices that incorporate vibration elements that connect to the outside of the leg and heel. One system includes a wearable system that may enhance human balance in gait and prevent foot injury through neurological stimulation of the foot via electrodes or vibrational actuators that are disposed in or on a wearable platform, such as in the insole or as removable shoe inserts. Many of these systems are designed to stimulate the Meissner corpuscles, but stimulating the Meissner corpuscles has not always been found effective. Meissner corpuscles only exist in the skin.

### SUMMARY OF THE INVENTION

This summary is provided to introduce a selection of concepts that are further described below in the Detailed Description. This summary is not intended to identify key or essential features of the claimed subject matter, nor is it intended to be used as an aid in limiting the scope of the claimed subject matter.

In general, an apparatus for treating neuropathy symptoms in a patient may comprise a platform comprising a base and a top plate supported by the base and configured to allow a patient to place their feet thereon. A vibrational actuator may be carried by the base and connected to the top plate and configured to vibrate the top plate when a patient has their feet thereon. A controller may be connected to the vibrational actuator and configured to operate the vibrational actuator to impart a vibrating force to the top plate when a patient has their feet thereon and stimulate the Pacini corpuscles to impart a neurological feedback response that dilates blood vessels of the feet and legs.

The controller may be configured to operate the vibrational actuator to impart a vibrating force to the top plate at a frequency between about 80 Hz and 2,000 Hz. The controller may be configured to operate the vibrational actuator to cycle on and off in about 25 to 35 second intervals. The base may include isolators that support the top plate for vibrational movement when a patient has their feet thereon. The isolators may comprise four circular isolators mounted on the base that support the top plate for vibrational movement. The base may comprise a housing having an interior compartment in which said vibrational actuator, controller and isolators are contained, and a power supply contained in the interior compartment and connected to said vibrational actuator and controller. The housing may be substantially rectangular configured and include side walls and said top plate is substantially rectangular configured and dimensioned larger than the base such that the edges of the top plate overly the upper edge of the side walls of the base.

In an example, the platform defined by the base and the top plate may be substantially rectangular configured and about 12 by 12 inches and four inches high. The controller may include a frequency adjustment circuit to allow adjustment of the frequency of the vibrating force imparted to the top plate. The frequency adjustment circuit may be configured to adjust automatically the frequency of the vibrating force imparted to the top plate. The frequency adjustment circuit may include a manual adjustment to allow a patient to adjust manually the frequency of the vibrating force imparted to the top plate. The vibrational force may impart a neurological feedback response that aids to alleviate pain associated with neuropathy symptoms in the patient.

### DESCRIPTION OF THE DRAWINGS

Other objects, features and advantages of the present invention will become apparent from the detailed description of the invention, which follows when considered in light of the accompanying drawings in which:

FIG. 1 is a front perspective view of the apparatus for reducing symptoms associated with neuropathy or other sensation deficits and sensation dysfunction in the lower extremity in a patient in accordance with a non-limiting example.

FIG. 2 is a rear perspective view of the apparatus of FIG. 1.

FIG. 3 is another perspective view of the apparatus of FIG. 1 showing by hidden lines the base.

FIG. 4 is a front elevation view of the apparatus of FIG. 1 showing by hidden lines the base.

FIG. 5 is a rear elevation view of the apparatus of FIG. 1 showing by hidden lines the base.

FIG. 6 is an isometric view of the base.

FIG. 7 is a plan view of the base showing in block outline the controller, the vibrational actuator, power supply and isolators.

FIG. 8 is a side sectional view of the base taken along line 8-8 of FIG. 7.

FIG. 9 is an isometric view of the apparatus of FIG. 1 without the top plate and showing by hidden lines the base.

FIG. 10 is an isometric view of the top plate.

### DETAILED DESCRIPTION

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Like numbers refer to like elements throughout.

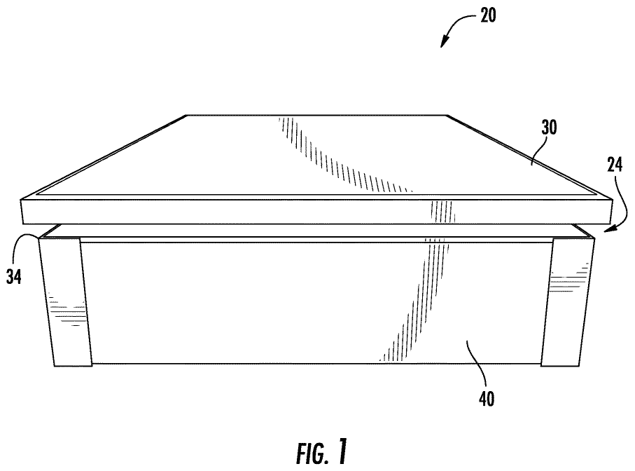

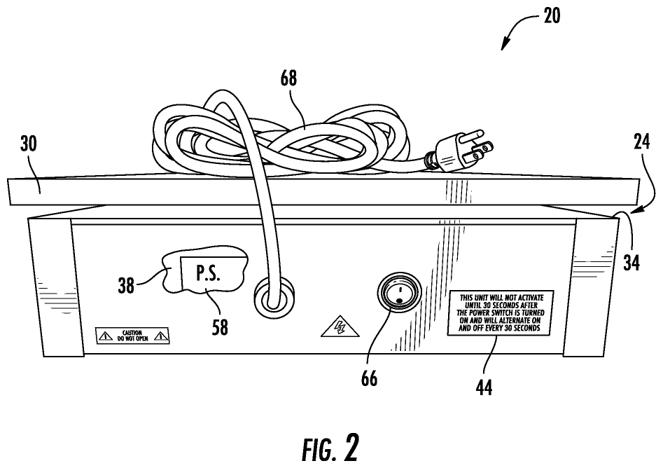

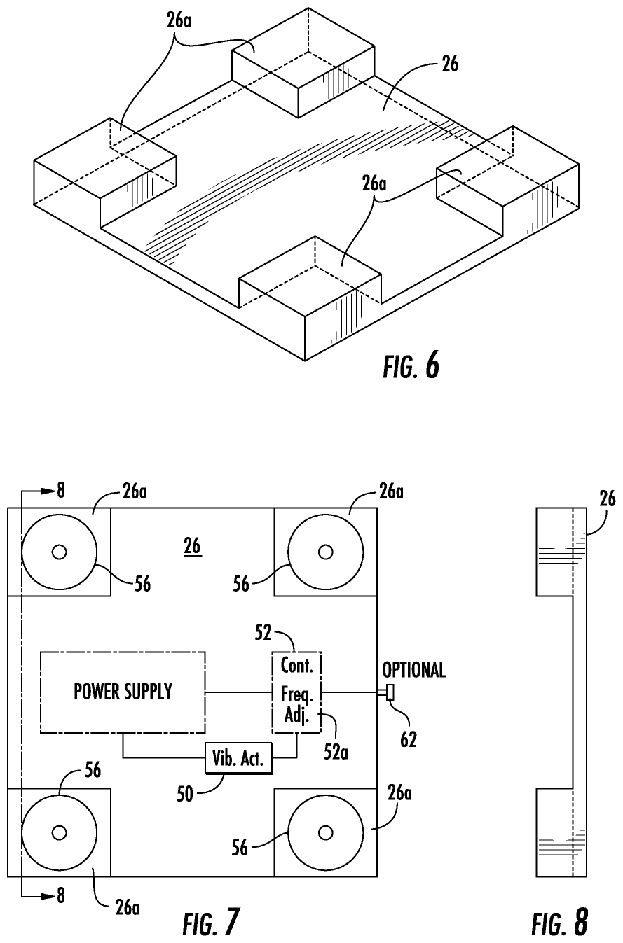

Referring now to FIGS. 1 and 2, there are illustrated generally at 20 perspective views of the apparatus for reducing symptoms associated with neuropathy such as pain or other sensation deficits and sensation dysfunction in the lower extremity in a patient. As illustrated, the apparatus 20 includes a platform 24 having a base 26 as shown in FIGS. 3-9. The plan view of the base is shown in FIG. 7 and an isometric view and side sectional view shown in respective FIGS. 6 and 8. A top plate 30 operates as a top cover and is shown in FIGS. 3-5 and 10, and supported by the base 26 on isolators 56 shown in FIG. 7 and as explained in greater detail below. The isolators 56 are positioned at the rectangular supports 26a of the base at each corner and may be configured as vibration absorbing pads and engage the top plate 30. The top plate 30 is configured as a planar plate to allow a patient to rest or place their feet thereon. The top plate 30 may include an adhesively attached covering as shown at 30a in FIG. 1 to prevent the patient from sliding on the top plate. The patient may stand or sit down when using the apparatus 20. For neuropathy, the patients may be sitting down in a chair with their feet on the top plate 30, or in some cases, may stand on the top plate in operation. For stimulating balance, the patient is normally standing on the top plate 30.

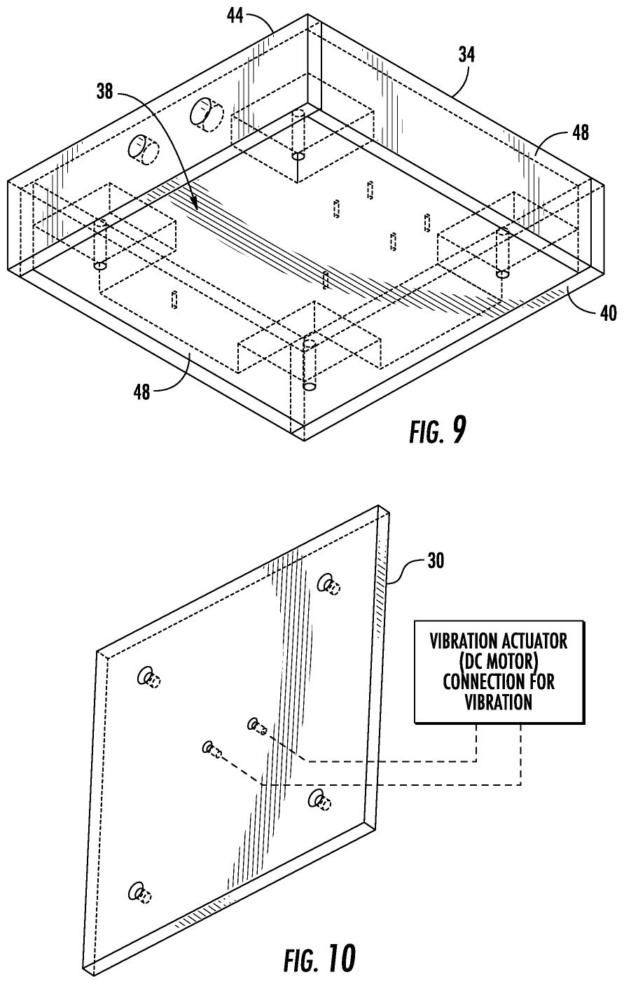

The base 26 is formed as a support for different side walls and is substantially rectangular configured (FIGS. 6-8). The base 26 and different side walls form a housing 34 having an interior compartment 38 as shown in the perspective view of the apparatus 20 in FIG. 3 and in FIG. 9 where the top plate 30 is not shown. The housing 34 is formed by the base 26 and front wall 40, rear wall 44, and two opposing side walls 48. The top plate 30 is substantially rectangular configured and configured larger than the rectangular configured base 26 such that the edges of the top plate 30 overly the upper edges of the front, rear and side walls 40,44,48 attached to the base, as shown in FIGS. 1 and 2, where the periphery of the top plate extends slightly over the upper or top side edge of the front, rear and side walls of the base.

The platform 24 defined by the base 26, walls 40,44,48 and top plate 30 in an example is about 12×12 inches as defined by the dimensions of the top plate and about 4 inches high. Each wall 40,44,48 may be about 11.5 inches long and about 3 inches high and about 0.5 inches thick and positioned on the outer perimeter of the base 26 to form the housing 34 and define the interior compartment 38 as shown in FIGS. 3 and 9. The base 26 may be about 10.5 inches square and with the walls 40,44,48 define the interior compartment 38 while the isolators 56 help space the top plate 30 from the upper edges of the walls.

The top plate 30 is engaged by the vibrational actuator 50 (FIG. 7) that imparts the vibration to the top plate. Two general types of materials may be used for the platform 24, but other materials may be used. For example, the base 26 may be made of low density PVC and the top plate 30 may be made from high density PVC since the top plate must withstand vibration and a patient may stand on that top plate. The base 26 and front, rear and side walls 40,44,48 may be held together by high strength adhesive, or mechanical screws or other fastening techniques, or may be molded as one piece such that the base includes the front, rear and side walls.

In an example, the base is about 11.5 inches square and the top plate 30 is about 12 inches square and overhangs the front, rear and side walls 40,44,48. The vibrational actuator 50 (FIG. 7) is carried by the base 26 and operatively connected to the top plate 30 and configured to vibrate the top plate, for example, when a patient stands thereon or the patient is sitting and rests their feet thereon. A controller 52 is connected to the vibrational actuator 50 and configured to operate the vibrational actuator at a selected frequency of vibration and impart a vibrating force to the top plate 30 when a patient has their feet thereon and stimulate the Pacini corpuscles to impart a neurological feedback response that dilates blood vessels of the feet and legs and lower extremity that are in close contact with the top plate when a patient stands or has their feet resting thereon. There is evidence that the vibration from the top plate 30 acts to dilate vessels in the legs and the lower extremities besides the feet.

The Pacini corpuscles, also referred to as the Pacinian corpuscles or lamellar corpuscles, are one of the four major types of mechanoreceptor cells. They have nerve endings in the skin, bones and some organs, and are responsible for sensitivity to vibration and pressure and typically respond to sudden disturbances or vibration. These corpuscles are usually larger and fewer in number than the Meissner corpuscles and other Merkel cells and Ruffini's corpuscles. They have lamellae that sense vibration and press on a membrane of a sensory neuron and cause it to bend or stretch. When these lamellae are deformed resulting from either pressure or release of pressure, a potential is created as the plasma membrane is physically deformed at the receptive area of the neuron, making it “leak” sodium ions.

The nerve impulses are action potentials that are formed by pressure-sensitive sodium channels. Signals generated by the Pacini corpuscles travel along a completely different nerve pathway to the brain than signals generated by the Meissner corpuscles. The Pacini corpuscles travel along fast acting highly myelinated nerves that traverse through the Posterior Columns which is highly associated with balance of the human body. The Meissner corpuscles are less myelinated, slower and do not travel through the Posterior Columns.

The Pacini corpuscles differ from Meissner corpuscles based on their morphology, distribution, and response threshold. The onion-like capsule in a Pacini corpuscle has an inner core and membrane lamellae, which are separated from the outer lamellae by a fluid-filled space with one or more adapting afferent axons at the center of the structure. This capsule acts as a filter allowing only transient disturbances such as at 250 to 350 Hz to activate nerve endings. Pacini corpuscles exist in the skin, bones, and some organs.

The controller 52 (FIG. 7) operates as a speed controller for an electrical motor that forms part of the vibrational actuator 50, and is configured to operate the vibrational actuator and impart a vibrating force at a frequency between about 80 Hz and 2,000 Hz. The inventors have found that at this range of frequencies, the Pacini corpuscles are stimulated and impart a neurological feedback response that dilates the blood vessels of the feet and lower extremity that are in close contact with top plate when the patient stands thereon or places their feet on the top plate while seated. This is a surprising result considering that most skilled in the art consider vibrations of 250 to 350 Hz to be optimum to stimulate Pacini corpuscles. The inventors have discovered that ranges above 200 Hz and as high a range as 350 Hz to 2,000 Hz are more beneficial for some patients. Each patient may respond to a different frequency range, but it has been found that the 80 Hz to 20,000 Hz frequency range is beneficial, and the higher frequency range above 350 Hz and 400 Hz to 2,000 Hz is beneficial, as compared to what those skilled in the art usually considered an upper limit of 350 Hz.

In an example, the controller 52 operating as a speed control circuit also includes a frequency adjust circuit 52a such as programmed instructions or other firmware to adjust the frequency of the vibrational actuator 50, which connects to the top plate 30, and thus, adjust the vibration of the top plate. The controller 52 may be configured to operate the vibrational actuator 50 and cycle on and off the vibrational actuator in different timed intervals, such as 25 to 35 second intervals. In an example, 30 second intervals has been found advantageous for most patients. This cycling in this example time period prevents the nervous system from adapting and ignoring the stimulation. The example 30 second time interval has been found advantageous, but may range in another example from 20 to 40 second intervals, and in another example, from 25 to 35 second intervals as noted before. The base 26 includes the isolators 56, which may be configured in different examples, such as circular configured isolators that may include vibration isolation (or absorption) pads that operate similar to shock absorbers that support the top plate 30 and dampen vibrational movement to the components in the interior compartment 38 when a patient stands or rests their feet on the top plate. In an example, the isolators 56 may include four circular configured isolators formed or mounted in the base 26 at each rectangular support corner and support the top plate 30 during its vibrational movement.

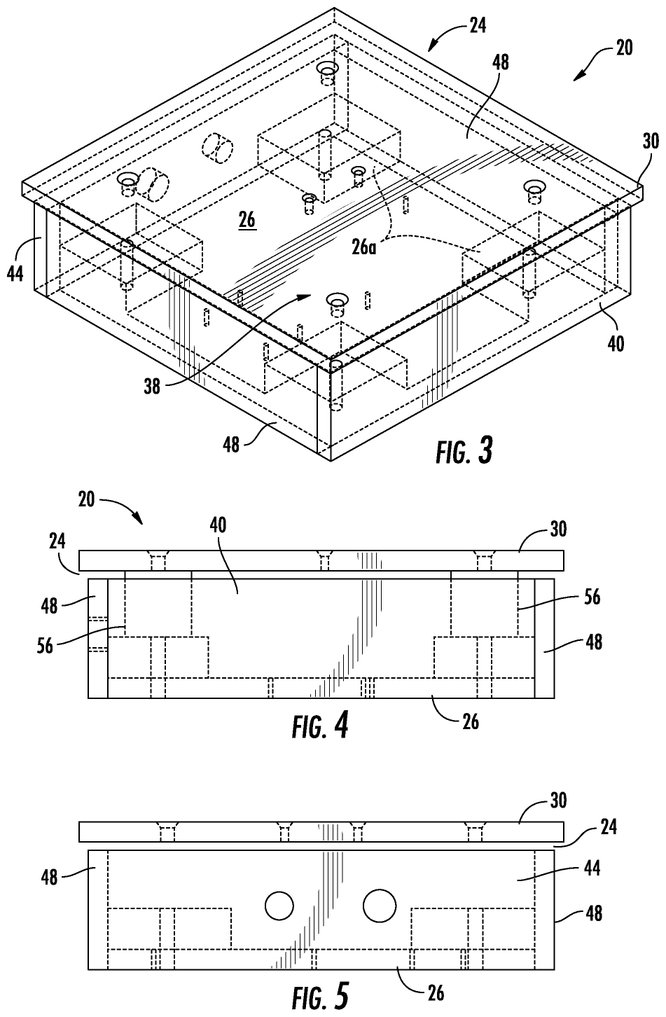

The housing 34 includes the interior compartment 38 in which the vibrational actuator 50, controller 52, isolators 56, and power supply 58 are contained together with other components. The isolators 56 are positioned at the rectangular support 26a at four corners of the base 26 (FIG. 7) and operate as support structures for the top plate 30 and include rubber or other shock absorbing mounts that help support the top plate and prevent excess vibration to components supported on the base such as the controller 52, vibrational actuator 50, and a power supply 58 that powers the controller and vibrational actuator. Isolators 56 are shown schematically by the dashed lines in FIG. 4 and engage the top plate 30 and base 26 at the rectangular support 26a.

In an example, the controller 52 includes its frequency adjustment circuit 52a to adjust the frequency of the vibrating force imparted to the top plate 30, which may be accomplished automatically depending on preprogrammed instructions. This is advantageous because different levels of neuropathy or different symptoms may best be treated at a range of vibrating forces at different frequencies. For example, one patient with a certain condition of peripheral neuropathy in the feet may find that their treatment works best when the vibrational actuator 50 imparts a vibrating force at a frequency between about 1,500 to 1,600 Hz, while another patient may find that their treatment is better effectuated when the vibrating force is at a frequency between 900 to 1,100 Hz. The controller 52 may include a logic circuit or associated software and/or other circuits that automatically adjust the frequency over time.

In some cases, the patient may find that they desire to manually adjust and vary the frequency over time. In that example, a separate and optional manual frequency adjustment knob 62 (shown figuratively in FIG. 7) connects to the controller and may extend outward from a wall that is attached to the base 26 to allow a patient to adjust manually the frequency of the vibrating force imparted to the top plate 30. In this example, as noted before, the manual frequency adjustment is an optional function.

The base 26 and housing 34 formed from the front, rear and side walls 40,44,48 may be formed from a hard plastic, aluminum, or other metal or plastic material. As shown in FIG. 2, the on/off switch 66 may connect directly to the power supply 58, and in this example, the power supply includes a power cord 68 for connecting to common household outlets and power at 120 volts, which may be used to operate the vibrational actuator 50 and controller 52. In some embodiments, batteries may be incorporated within the interior compartment 38 and power the apparatus 20.

It was found that the Pacini corpuscles and their corresponding neurological pathway to the brain are usually not stimulated below about 100 Hz, and the higher vibration above 100 Hz (and in some cases 80 Hz) includes a neurological feedback response that dilates the blood vessels in the areas in close contact with the top plate 30. In operation, when a patient stands or has their feet resting on the top plate 30, the vibration causes a distinct visual observation of pigment change in the skin that was a red or purple color. There is also a significant improvement in balancing in many patients who have used the apparatus 20 more than a few weeks. Most patients also report increased feeling in areas that were “numb” before the application of vibration from the top plate 30.

The apparatus 20 with its platform 24 and various components may be manufactured in accordance with ASTM standards and be formed with ISO standards. The power supply 58 may be configured as a low voltage power supply 58 as the power source and one or two circuit boards for the controller 52 and other components, including the vibrational actuator 50 and related components.

The power supply 58 in an example is a NP-60-12 volt power supply having an input of 120 volts and 12 volt output. The power supply 58 is designed for specifications in operating the platform. The vibrational actuator 50 may include a 12 volt DC brushless motor built to specifications for the apparatus 20. In an example, the motor includes an output shaft having an offset load or weight such as an eccentric rotating mass creating an imbalance and centripetal force. The motor may be mounted to the underside of the top plate 30, via fasteners that may extend into the two centrally located fastener holes in the top plate as shown schematically in FIG. 10 by the block legend indicating “vibration actuator (DC motor) connection for vibration”. In some examples, it may operate by a connection to the top plate 30 for vibrating the top plate. The imbalance induced from the offset load on the motor shaft creates the vibration in the top plate 30, which varies in frequency as understood when motor speed on its output shaft varies. Force may be dependent on the size of the mass and speed of the motor. The speed control as part of the frequency adjustment circuit 52a may include a low voltage speed control circuit used to calibrate the required frequency of the vibration. The apparatus 20 may include a 12 volt timing circuit that is used to control the intermittent on or off timing, such as 25 to 35 seconds, and in an example, 30 second timing.

The isolators 56 as noted before may include four customer made circular configured rubber isolators placed at each corner of the base 26 and engage the top plate 30. The isolators 56 may be attached by screws (not shown) that extend from the upper surface of the top plate 30 to the isolators to retain the top plate to the isolators. The isolators 56 isolate the vibration from the top plate 30 to the base 26, where the different components are installed. In an example, the isolators 56 may include female threads on each side with a 5/16-18 thread. They have in an example a hardness of A-shore 70. The top plate 30 also includes two centrally located screw holes that may receive screws or other fasteners to help secure the top plate to the vibrational actuator 50.

Many modifications and other embodiments of the invention will come to the mind of one skilled in the art having the benefit of the teachings presented in the foregoing descriptions and the associated drawings. Therefore, it is to be understood that the invention is not to be limited to the specific embodiments disclosed, and that the modifications and embodiments are intended to be included within the scope of the dependent claims.

1. A apparatus for treating neuropathy symptoms in a patient, comprising:
a platform comprising a base and a top plate supported by the base and configured to allow a patient to place their feet thereon;
a vibrational actuator carried by the base and connected to the top plate and configured to vibrate the top plate when a patient has their feet thereon; and
a controller connected to the vibrational actuator and configured to operate the vibrational actuator to impart a vibrating force to the top plate when a patient has their feet thereon and stimulate the Pacini corpuscles to impart a neurological feedback response that dilates blood vessels of the feet and legs.

2. The apparatus of claim 1 wherein the controller is configured to operate the vibrational actuator to impart a vibrating force to the top plate at a frequency between about 80 Hz and 2,000 Hz.

3. The apparatus of claim 1 wherein the controller is configured to operate the vibrational actuator to cycle on and off in about 25 to 35 second intervals.

4. The apparatus of claim 1 wherein the base includes isolators that support the top plate for vibrational movement when a patient has their feet thereon.

5. The apparatus of claim 4 wherein the isolators comprise four circular isolators mounted on the base that support the top plate for vibrational movement.

6. The apparatus of claim 4 wherein the base comprises a housing having an interior compartment in which said vibrational actuator, controller and isolators are contained, and a power supply contained in the interior compartment and connected to said vibrational actuator and controller.

7. The apparatus of claim 6 wherein the housing is substantially rectangular configured and includes side walls and said top plate is substantially rectangular configured and dimensioned larger than the base such that the edges of the top plate overly the upper edge of the side walls of the base.

8. The apparatus of claim 1 wherein said platform defined by the base and the top plate is substantially rectangular configured and about 12 by 12 inches and four inches high.

9. The apparatus of claim 1 wherein the controller includes a frequency adjustment circuit to allow adjustment of the frequency of the vibrating force imparted to the top plate.

10. The apparatus of claim 9 wherein the frequency adjustment circuit is configured to adjust automatically the frequency of the vibrating force imparted to the top plate.

11. The apparatus of claim 9 wherein the frequency adjustment circuit includes a manual adjustment to allow a patient to adjust manually the frequency of the vibrating force imparted to the top plate.

12. The apparatus of claim 1 wherein the vibrational force imparts a neurological feedback response that aids to alleviate pain associated with neuropathy symptoms in the patient.

13. A apparatus for treating neuropathy symptoms in a patient, comprising:
a platform comprising a base and a top plate supported by the base and configured to allow a patient to place their feet thereon;
a vibrational actuator carried by the base and connected to the top plate and configured to vibrate the top plate when a patient has their feet thereon;
a controller connected to the vibrational actuator and configured to operate the vibrational actuator to impart a vibrating force to the top plate when a patient has their feet thereon and stimulate the Pacini corpuscles to impart a neurological feedback response that dilates blood vessels of the feet and legs, wherein said controller is configured to operate the vibrational actuator to impart a vibrating force to the top plate at a frequency between about 80 Hz and 2,000 Hz and operate the vibrational actuator to cycle on and off at about 25 to 35 second intervals; and
isolators carried by the base that support the top plate for vibrational movement when a patient has their feet thereon.

14. The apparatus of claim 13 wherein the base comprises a housing having an interior compartment in which said vibrational actuator, controller and isolators are contained, and a power supply contained in the interior compartment and connected to said vibrational actuator and controller, and the isolators comprise four circular isolators mounted in the interior compartment at corners thereof that support the top plate for vibrational movement.

15. The apparatus of claim 14 wherein the housing is substantially rectangular configured and includes side walls and said top plate is substantially rectangular configured and dimensioned larger than the base such that the edges of the top plate overly the upper edge of the side walls of the base.

16. The apparatus of claim 13 wherein said platform defined by the base and the top plate is substantially rectangular configured and about 12 by 12 inches and four inches high.

17. The apparatus of claim 13 wherein the controller includes a frequency adjustment circuit to allow adjustment of the frequency of the vibrating force imparted to the top plate.

18. The apparatus of claim 17 wherein the frequency adjustment circuit is configured to adjust automatically the frequency of the vibrating force imparted to the top plate.

19. The apparatus of claim 17 wherein the frequency adjustment circuit includes a manual adjustment to allow a patient to adjust manually the frequency of the vibrating force imparted to the top plate.

20. The apparatus of claim 13 wherein the vibrational force imparts a neurological feedback response that aids to alleviate pain associated with neuropathy symptoms in the patient.
